(19) United States
(12) Patent Application Publication
(10) Pub. No.: US 2008/0311230 A1
(43) Pub. Date: Dec. 18, 2008

(54) Preparation of Artemisia to treat human cancer, autoimmune disease, IgA-Nephropathy, and to counteract weight loss in cancer patients

(76) Inventor: Harun A. Omer, (US)

Correspondence Address: Harun Omer, Stelleacker 18, Rheinfelden, 79618, DE

(21) Appl. No.: 11/764,205

(22) Filed: Jun. 17, 2007

(51) Int. Cl. A61K 36/282

(52) U.S. Cl. 424/740

(57) ABSTRACT

In human cancer, reduced appetite and weight loss are serious symptoms and are difficult to manage. These symptoms not only effect the quality of life, but also reduce the tolerance and response to anti-cancer drugs, thereby effecting the prognosis and survival to disease. These symptoms are caused, among others, by overactive immune system of the body. Anti-cancer drugs that were effective in the beginning start losing their efficacy, and are less tolerated. Also in autoimmune diseases, the immune system is overreacting. This herbal invention, based on Artemisia species, ginger root and large cardamom, each targeting at different factors involved in weight loss and autoimmune diseases, is effective in improving appetite, thus counteracts the development of resistance to anti-cancer drugs, and helps in the cure of autoimmune diseases, IgA-Nephropathy, which has no cure so far.

### REFERENCES

1) Nadkarni A. K. (Ed.) Indian Materia Medica. Popular Book Depot Bombay Dhootapapeshwer Prakashan Ltd. Panvel

2) Dastur J. N. (Ed.) Useful Plants of India and Pakistan. D. B. Taraporewala sons & Co. Ltd. Treasure House of Books, Bombay, India.

3) Glossary of Indian Medicinal Plants. Pages Council of Scientific & Industrial Research India (Ed.)

4) Hoppe H. A. (Ed.) Drogenkunde; de Gruyter Verlag

5) Encyclopaedia of Common Natural Ingredients Used in Food, Drugs, and Cosmetics

6) British pharmacopoeia

7) Handbuch der Drogenkunde. Ed.: F. Berger Verlag W. Maudrich, Vienna.

8) Pharmazeutische Biologie. Ed.: F. Deutschmann, B. Hohmann E. Sprecher, E. Stahl. G. Fischer Verlag, Stuttgart, New York.

10) Handbuch des Arznei- und Gewürzpflanzenbaus. Deutscher Bauernverlag, Berlin.

11) Illustrierte Flora von Mitteleuropa. Verlag Paul Parey, Berlin, New York.

12) Hagers Handbuch der Pharmazeutischen Praxis Für Apotheker, Arzneimittelhersteller, Ärzte und Medizinalbeamte Ed. P. H. List and L. Hörhammer, Springer-Verlag Berlin, Heidelberg, New York

### STATEMENT AS TO THE RIGHTS OF INVENTION

Harun Omer Ph.D. is the sole inventor of this patent. The invention was not sponsored by any government organisation.

### BACKGROUND

Plants have been the basis of treatment of human diseases from time immemorial. Every country in the world has its list of herbal remedies for various health disorders. The foundations of the modern drug industry are essentially based on the isolation of active compounds from plants, which were developed further synthetically to obtain more suitable analogues. Artemisin from the Artemisia herb is the recent example for a treatment of multi-drug resistant malaria.

According to the present drug-receptor theory, the compatibility of the structure of a drug with the receptor is vital for its pharmacological activity. Following this principle, most of the modern drugs have been invented by synthetic modification of active molecules from natural or other sources. Such drug-receptor interaction can be simulated in the laboratory on animal and cell models, and the best possible structure for maximum activity are discovered. In this exercise, a large number of structural analogues are synthesized and further tested for the desired pharmacological or biological activity. The main purpose of this exercise is to determine the most suitable structure for maximum activity. Most drugs which have been developed from such exercises are not completely compatible with the requirements of the receptor sites, therefore they have limitations in therapeutic management and simultaneously exhibit toxic side effects.

The plant on the other hand is capable of producing a wide range of analogues, at least one of which possesses the desired receptor compatibility. The other related compounds appear to exercise a synergistic effect on the pharmacological activity of the compatible compound and at the same time suppress its toxic effects. However, a major drawback in using plant material in their crude form is that the amount of plant material needed for therapeutical benefit is quite high, sometimes even up to 10 g a day or more. Such quantities cannot be conveniently converted into suitable dosage forms. A solution to this problem is to find herbs that exhibit a synergetic effect when combined with each other, leading to a preparation which is clinically effective and is at the same time in a convenient administrable dosage form.

Many plants examined for critical research for the development of new drugs are toxic for human use. Therefore testing their anti-cancer or their other medically usefully activity, establishing their effective dose and their safety is a long and difficult process. A major problem to be solved by this invention was to select plants, either absolutely non-toxic to humans or at least whose toxicity was well established with approved doses. The process, according to this invention, also seeks to provide a beneficiated plant preparation, which contains all pharmacologically active chemicals in their original natural state and proportions instead of concentrating on one of the metabolites.

With this invention, we succeeded in finding effective and at the same time well-known, widely used and safe plants that can be employed in combination with conventional available chemotherapies to treat the resistant and progressing cancer and autoimmune diseases. We discovered reasons to believe that plants of the artemisia species, ginger, and large cardamom meet the requirements of this invention. This invention is the first to describe a combination of herbal therapy with conventional evidence based chemotherapy to treat some of the most difficult diseases.

Of the 800 or more articles on PubMed's data base on Artemisia, only a few deal with cancer and autoimmune diseases. The reports are mainly on cell or animal models. Of these, none describes clinical trials of Artemisia in humans for the treatment of therapy resistant cancer. Similar is the case with ginger and large cardamom. The inventor worked with many doctors for several years to find out what works and what doesn't in the most difficult, therapy resistant cases of cancer and autoimmune diseases in humans. The claims made in this invention do not rely on cell model studies or animal studies, but on clinical trials that were planned and carried out to support the claims made in this invention.

### SUMMARY OF THE INVENTION

This herbal invention of Artemisia is unique to treat autoimmune diseases and human cancers that are progressing in spite of continued conventional standard treatments. Hereby the invention should be applied preferably in combination with the conventional standard treatment, even though the conventional standard treatment is no longer effective or is showing signs of failure. The invention also provides cure to IgA-Nephropathy, a kidney disease with no satisfactory treatment available so far. Hodgkin as well as Non-Hodgkin lymphomas can also be treated with this invention where standard treatment is showing signs of failure, provided the standard conventional therapy is continued. The invention also counteracts significantly the weight loss and nutritional deficiencies that have negative impact on cancer patients.

### Description

Following is a short description of the herbs that constitute this invention.

### The Artemisias:

We have included in our claim all major artemisia species in common traditional use in Asia, Africa, China, and Europe. Artemisia absinthium (wormwood), A. annua (sweet wormwood), A. afra (African wormwood), A. capillaries (Korean wormwood), A. vulgaris (common wormwood) and A. asiatica (Asian wormwood) are, as far as their ethnopharmacology and ethnobotanical appearance is concerned, very similar. All belong to the same species and enjoy widespread and similar uses in traditional medicine in Africa (A. afra), in Asia (A. asiatica), in China (A. annua), far east (A. capillaries) and Europe (A. absinthium). Common to them is their traditional use against malaria and liver diseases. Also common to them are claims of their anti-inflammatory, antifungal, and antipyretic activity. They all contain lactones such as arabsin, artabin, ketopelenolide, and others related to santonin. They all contain thymol and carvacrol as well as other phenolic compounds with potent antioxidant and radical scavenging activity. The medicinal or active components for the treatment of malaria—artemisin, is present in all of them in varying amounts. Their bitter taste is caused by the glucosides absinthin and anabsinthin, and several related compounds, that also vary in amount. For example A. absinthium, the common wormwood, is more bitter than A. annua because A. absinthium contains a higher concentration of the bitter glucosides. For that reason, A. annua is called the sweet wormwood.

The major components of artemisia oils include chamazulene, nuciferol butanoate, nuciferol propionate, and caryophyllene oxide. Their essential oils also contain a large amount of similar aromatic compounds and a low level of oxygenated monoterpenes. Again all these constituents vary from species to species and crop to crop.

All these artemisias contain monoterpene-thujone derivatives that have toxic effects on the central nervous system and cause convulsion when taken in large quantities. The quantity of thujones differs not only from species to species but also from crop to crop.

Because of these minor differences one speaks today not only of African artemisia (A. afra), Asian artemisia (A. asiatica), Chinese artemisia (A. annua) but also of French artemisia, Italian artemisia, German artemisia, Indian artemisia, Croatian artemisia etc. etc. Since these are only minor differences in quantity and not differences in quality, some ethno-pharmacologists are even of the opinion that they are the same species, with the small changes caused as a result of their geographical and climatic distribution.

Artemisia Dose: Their classical use is at a dose of 2 to 5 g per day. In 1979, the FAO/WHO Codex Committee on Food Additives restricted the use of alpha- and beta-thujones to the following maximum levels in final products ready for consumption: 0.5 ppm in food and beverages, 10 ppm in alcoholic beverages containing more than 25% alcohol, 5 ppm in alcoholic beverages containing less than 25% alcohol, and 35 ppm in bitters.

Large Cardamom (Amomum subulatum; Zingiberaceae)

Large cardamom or the fruit of Amomum subulatum Roxb, (Zingiberaceae) commonly known as “Heel kalan” or “Bari Ilaichi” belongs to the family of ginger and is mentioned in pharmacopoeia books of European countries and many others around the world. It is widely used as spice in the Indian sub-continent. In traditional medicine, it is used to relieve gastrointestinal disorders, but there is no clinical evidence to support dose recommendations. Large cardamom is non-toxic and safe for human. Its use in cancer or for autoimmune diseases is unknown.

Ginger root (Zingiber offinale rhizome; Zingiberaceae)

The ginger root (Zingiber offinale rhizome; Zingiberaceae) is mentioned in pharmacopoeia books of European countries and many others around the world. It was first cultivated in Asia and has been used as a medicinal herb for at least 2,000 years. Medicinal reference to ginger root appears in early Sanskrit and Chinese text as well as in ancient Greek, Roman, and Arabic medical literature. Ginger root was introduced to the Americas by the Spaniards in the 18^{th} century. The rhizome was being used to reduce irritant effects of various medicines on the stomach. In Western herbal medicine ginger root is used as circulatory stimulant, to treat colds and flu, and for dyspepsia, colic, nausea, vomiting, gastritis, and acute colitis.

Ginger root contains about 1-3% volatile oils, which give the plant its distinctive aroma, and from 1 to 2.5% gingerols and shogaols, a non-volatile oily liquid consisting of homologous phenols, which give ginger root its pungent or hot quality. Gingerols and shogaols are phenolic compounds (made up of phenyalanine, malonate, and hexanoate). Phenololic compounds are a large class of water-soluble organic compounds, which have an aromatic ring that generally contains one or more hydroxyl substitutes. Several gingerols of various chain-lengths are present in ginger, the most abundant being 6-gingerol. The gingerols and shagaols are also the chemical constituents, which are responsible for ginger's anti-emetic effects.

The pain killing properties of ginger root in joint diseases have been known and valued for centuries. In the past 25 years, many laboratories have provided scientific support for the long-held belief that ginger root contains constituents with anti-inflammatory properties that help reducing the inflammation of joints. The original discovery of ginger root's inhibitory effects on prostaglandin biosynthesis in the early 1970s has been repeatedly confirmed. This discovery identified ginger root as an herbal medicinal product that shares pharmacological properties with non-steroidal, anti-inflammatory drugs such as Aspirin and Ibuprofen. These properties have nothing in common with our invention, as neither Aspirin nor Ibuprofen can heal or reverse the progressing rheumatic polyarthritis, which is an autoimmune disease. In other autoimmune diseases, such as regional colitis, anti-inflammatory drugs (e.g. Aspirin and Ibuprofen) are even contra-productive. There is no recent clinical evidence to support dose recommendations for ginger root. This root is non-toxic and safe for human use. A few anticancer studies of ginger are limited to laboratory cell lines or animal models, no human studies have been reported.

Following is a brief description of diseases that benefit from this invention:

### Autoimmune Diseases:

Autoimmune diseases are progressive chronic diseases of various causes and have a wide spectrum of disease severity. Many patients run a course of intermittent relapses and remissions with an overall pattern of slowly progressive organ failure. They have some common symptoms that include pain and an inflammation of one or more organs such as muscles and skin, joints, kidney, eyes, gastro-intestinal tract etc. and they have common treatment strategies with similar drugs. At present, the standard treatment are steroids combined with immune modifying drugs such as penicillinamine, cyclophosphamide, methotrexate, gold salts, azothioprine, levamisole and recently the so called TNF-alpha inhibitors. Almost all have severe side effects and are toxic. Moreover, their effects are mainly of short duration and there are many disadvantages in the use of these drugs over extended periods of time. In addition, many recent drugs are costly and have low benefit-risk ratio. The ideal drug to modify the progress of the disease has not yet been found.

### IgA Nephropathy

Synonyms: Immunoglobulin A nephropathy, Berger disease, mesangial glomerulonephritis, mesangial proliferative glomerulonephritis

IgA nephropathy is a kidney disorder caused by deposits of the protein immunoglobulin A (IgA) inside the glomeruli within the kidney. These glomeruli normally filter wastes and excess water from the blood and send them to the bladder as urine. The IgA protein prevents this filtering process, leading to blood and protein in the urine and swelling in the hands and feet. At this stage the patient must go on dialysis or receive a kidney transplant. This disease accounts for about 10% of biopsies performed for glomerular disease in the United States. IgA nephropathy is now one of the most common cause of kidney failure in the world.

The pathology of immunoglobulin A (IgA) nephropathy was first described by Berger and Hinglais in 1968. By the early 1980s, long-term follow-up data illustrated that some patients with IgA nephropathy have a slow progression to end-stage renal disease (ESRD). The condition can lead to chronic renal insufficiency. Many other systemic diseases are sporadically associated with mesangial IgA deposition. Henoch-Schönlein purpura (HSP), a systemic illness, has been closely linked to IgA nephropathy. Other systemic diseases in which mesangial deposits of IgA are regularly observed include systemic lupus erythematosus, hepatitis, dermatitis herpetiformis, and ankylosing spondylitis. The characteristic pathologic findings by immunofluorescence microscopy of granular deposits of IgA and complement 3 (C3) in the glomerular mesangium suggest that this disease is the result of the deposition of circulating immune complexes leading to the activation of the complement cascade. Deposited IgA is predominantly polymeric IgA1, which is mainly derived from the mucosal immune system.

A class of blood pressure lowering drugs called ACE inhibitors protects kidney function for a limited period of time not only by lowering blood pressure but also by reducing the loss of protein into the urine. These drugs however cannot stop the course of the progressive disease. Corticosteroids have no curing effect. They may suppress the production of IgA for a short time but can have harmful side effects. In short there is no treatment for this disorder so far.

### Hodgkin and Non-Hodgkin Lympomas

Lymphomas are cancers of the lymphatic system—the body's blood-filtering tissues that help to fight infection and disease. Like other cancers, lymphomas, occur when cells divide too much and too fast. Growth control is lost, and the lymphatic cells may overcrowd, invade, and destroy lymphoid tissues and spread to other organs.

There are two general types of lymphomas: “Hodgkin's Disease” (named after Dr. Thomas Hodgkin, who first recognized it in 1832) and non-Hodgkin's lymphoma. The lymphatic tissue in Hodgkin's disease contains specific cells—Reed-Sternberg cells—that are not found in any other cancerous lymphomas or cancers. These cells distinguish Hodgkin's disease (HD) from non-Hodgkin's lymphomas (NHLs). Non-Hodgkins lymphoma (NHL) is a hetrogenous disease. Each year, there are approximately 50,000 new cases and almost 25,000 deaths from the disease in the United States. Unlike Hodgkins disease, NHL is comprised of approximately 10 different subtypes (in the Working Formulation) and 20 different disease entities in the Revised European-American Lymphoma Classification (REAL) system.

These subtypes are grouped into 3 biologic states—low grade, intermediate grade, and high grade lymphomas. Therapy is determined by several factors, including the biologic state of the lymphoma, the stage of lymphoma, the presence or absence of symptoms (e.g., weight loss, night sweats, organ dysfunction), and the overall general health of the patient.

A number of factors, including congenital and acquired immunodeficiency states, and infectious, physical, and chemical agents, have been associated with an increased risk for NHL. Infectious agents, such as viral infections (e.g., Epstein-Barr virus, human T-cell leukemia virus), and bacterial infections (e.g., heliobacter pylori) may be associated with the development of NHL. Additionally, physical and chemical agents such as pesticides, solvents, arsenate, and lead, as well as hair dyes, radiation exposure (high dose), and paint thinners may also increase the risk.

Non-Hodgkins lymphoma occurs more often in patients between the ages of 40 and 70.) Risk for disease recurrence and overall survival rate can be predicted by using an international prognostic index (IPI) which takes into account age, stage of disease, general health (also known as performance status), number of extra nodal sites, and presence or absence of an elevated serum enzyme named LDH (lactate dehydrogenase).

### Progressing Cancer

Cancer is the leading cause of death. Surgery, radiation and chemotherapy are the most widely used therapeutic modalities. In spite of all these measures the cancer growth continues in many patients and no anticancer therapeutic modality works. The chemotherapy that seemed to help initially stops working. A kind of resistance against this chemotherapy develops. The alternative is either to increase the dose of the already highly toxic chemotherapeutic agent and risk the life of the patient or give up all therapeutic attempts. In cancer, after chemotherapy, the immune system starts to over-react, resulting in the failure of chemotherapy after its initial success. This can be demonstrated by an increase in levels of immune-cytokines involved in the immune system.

### Weight Loss in Cancer Patients

All cancer patients will suffer from extreme weight loss and nutritional deficiencies at some stage of their disease. This is not only because the cancer growth is consuming a large portion of incoming energy. But the cancer is producing substances that suppress the feeling of hunger of the suffering patients. Studies have shown that routine use of artificial nutrition will greatly improve the outcome of the cancer treatment and longevity of life. According to current evidence, most organizations, including the American Society for Parenteral and Enteral Nutrition (A.S.P.E.N.), do not recommend the routine use of artificial nutrition for patients with cancer. Therefore there is a need for alternative methods to improve nutrition and the supply of energy.

### Immune-Modulation

Immune-modulation is a procedure that can alter the immunity by interfering with its functioning. Immune-modulation plays an important role in the treatment of many diseases. Autoimmune diseases such as rheumatoid arthritis, lupus, myositis, sklerodermia, colitis, Reiter's disease are some examples in which the immune system is over-reacting.

Most cancer patients and patients suffering from auto-immune diseases develop resistance to standard chemotherapy. This results in recurrence of their disease. A few immune-modulators are being investigated to overcome the problem of resistance. It is for example the notorious thalidomide and its derivatives. This drug, with extreme side effects, is being given out of frustration, along with standard chemotherapy to counteract the developing resistance to cancer drugs and to the drugs being used to treat autoimmune diseases. It is out of frustration as there are no satisfactory immune-modulators to counteract the developing resistance.

There is yet another alternative to modulate the immune system. This alternative is based upon employing our invention that suppresses the over-reactive immune system of cancer patients that have undergone the chemotherapy. From the examples of our invention it can be postulated that the invention works by immune-modulation.

### OBJECTS OF THE PRESENT INVENTION

The main objects of the present invention are summarized below:

1. Most cancer patients and patients with lymphomas and auto-immune diseases develop recurrent disease that eventually becomes resistant to standard chemotherapy. The object of this invention is to circumvent resistance to conventional chemotherapy of these diseases. This invention will increase the effectiveness of chemotherapy when added to standard chemotherapy treatment.

2. A second major object of the present invention is to provide treatment to those cancer patients and IgA Nephropathy where no effective treatment is available so far.

3. The third major object of the present invention is to improve nutrition of patients suffering from progressing cancer, as all cancer patients start loosing weight at some stage of their disease.

4. A further object of this invention is to provide a composition aforesaid that acts without exerting toxic side effects.

5. Still another object of this invention is to provide a disease-specific synergistic composition in convenient dosage form.

### DESCRIPTION

### Herbal Preparations

The beneficiated plant preparations was made according to the processes enunciated below.
1. Grinding
2. Extraction
3. Distillation

Grinding: This process comprises the steps of cleaning the plants to remove any foreign matter, bacteria, fungus, metals and fertilizers. Thereon grinding the plant to obtain a mass having particle size ranging from 0.0001-0.01 mm³, mixing them in the amounts that appear in the following examples, filling them in hard gelatine capsules or pressing them in tablets.

Extraction: When an extract has to be made, the grinded mass is subjected to one polar solvent followed by one non-polar solvent to obtain separate fractions of the plant extract soluble in the respective solvents and mixing the fraction so obtained to obtain the beneficiated plant extract. A typical procedure is as follows:

Example: Boil 10 g of powdered mass in a polar solvent for ten minutes, dissolve polar solvent soluble material to obtain a first solution and a first residue R1; filter the first solution from the first residue; evaporate the filtrate obtained from the first solution to remove the solvent and obtain a solute designated as fraction A from the powdered mass. Subject the first residue R1 to treatment with a second polar solvent, for twelve to thirty-six hours to obtain a second solution and a second residue R2; filtering the second solution from the second residue R2 to obtain a second filtrate; evaporating the second filtrate to remove its solvent and obtain a solute designated as fraction B from the powdered mass. Subject the second residue R2 to non-polar solvents for twelve to thirty-six hours to obtain a third solution and a third residue R3; filtering the third solution from the third residue R3 to obtain a third filtrate; evaporating the third filtrate to remove the solvent and obtain a solute designated as fraction C and homogeneously mixing the volatile fraction, with fractions A, B and C, to obtain the plant part extract. Dissolve the combination A+B+C in 10 ml 40% Alcohol so that each ml of this final extract contains extracted metabolites from an equivalent to 1 g of powdered herb.

Distillation: When distillation was to be done to prepare tinctures of the grinded mass is subjected to boiling with water, collecting the distillate in a cooled flask, then repeat the distillation by boiling with a polar and then a non polar solvent, collecting the distillate in separate cold flasks, combining the distillates of all three solvents, evaporate them and adding 40% alcohol to get the tincture.

Example: Boil 10 gm of powdered herb with 50 ml of distilled water, collect the distillate in a cooled flask (distillate A), when the water is completely distilled, repeat the distillation with 50 ml of polar solution, collect the distillate in the cooled flask (distillate B). When the polar solution is completely distilled, add 50 ml of non-polar solution and boil for some time, collect the distillate in cooled flask (distillate C) till this distillation is complete and no more polar solution is left in the residue. Combine the distillates A, B and C, evaporate the solutions of the distillate and dissolve them in 10 ml of 40% alcohol so that each ml of the final alcoholic solution (tincture) contains a distillate equivalent to 1 g of original powdered herb.

### Example of Doses:

| | |
|---|---|
| Dried herbal powder = | 3-6 tablets per day as 750 mg pressed tablets |
| Liquid Extract = | 2 ml three times a day (equivalent to 6 g of herb) |
| Distilled preparations | 2 ml three times a day (equivalent to 6 g of herb) |
| (Tinctures) = | |
| Powdered herbal | 6-9 capsules per day as 450 mg capsules of |
| mixtures = | size 00 equivalent to 2.7-4.0 g per day |

### EXAMPLES

The invention is illustrated with the following typical examples:

### Example 1

Distilled preparation (tincture) of A. absinthium to treat colon cancer with liver metastasis.

### Example 2

Powdered dried A. annua to treat multiple myeloma patients whose disease recurred in spite of chemotherapy and became resistant to available conventional treatments.

### Example 3

Distilled preparation (tincture) of A. absinthium and large cardamom to treat progressing uveal melanoma stage IV.

### Example 4

Extract of ginger root and A. capillaris to treat regional colitis—an autoimmune disease.

### Example 5

Powdered dried combination of A. vulgaris, or A. asiatica with ginger root and large cardamom in capsules to treat rheumatic polyarthritis—an autoimmune disease.

### Example 6

Powdered herbal combination of A. annua, ginger root and long cardamom in capsules to treat IgA Nephropathy

### Example 7

Powdered dried combination of A. absinthium, ginger root and large cardamom in the form of tablets to treat Gangliobalstoma cancer stage IV

### Example 8

Powdered dried combination of A. absinthium, ginger and large cardamom in capsules to treat Hodgkin and Non-Hodgkin lymphomas.

### Example 9

Powdered dried combination of A. absinthium, ginger and large cardamom to counteract weight loss and improve nutrition in cancer patients

### Example 1

Distilled preparation of A. absinthium to treat colon cancer with liver metastasis.

Invention 1: Distilled extract (tincture) of A. absinthium 3 ml given thrice a day for the treatment of progressing colon cancer (Dose 9 g/day)

Case Report: A male patient born on Apr. 22, 1954 was first diagnosed a colorectal carcinoma in May 2004 at a stage of uT3 cN0 cM0 G2. Before surgical removal he was treated with 3 cycles of Oxaliplatine/Capectabline and radiation at the University of Basel, Switzerland. Colon surgery was performed in August 2005. Histology staging showed ypT3 ypN1 R0. The surgery was followed by another five cycles of Oxaliplatine/Capectabline treatment. The patient was recovering. A complete body scan with MRT and PET revealed no cancer nodes at any place of the body. In February 2005 routine chest X-ray revealed three small nodes in the left lung of the size 2-3 mm. Two months later, in May 2005 another two nodes emerged, this time in the right lung of the size 3-4 mm. CT of the abdomen showed at the same time a single round metastasis of the 3.5 cm size. Chemotherapy was changed. Four cycles of Campto and Erbitux were given and the VI and VII segment of the liver, where the metastasis was found, surgically removed. Following treatment with Campto/Erbitux cycles reduced the sizes of lung metastasis initially, tumor marker CEA levels dropped to normal. However three months later, in August 2005 new nodes of metastasis were found in liver and lung. In spite of treatment with several cycles of Erbitux/Irinotecan the cancer was progressing as indicated by a steady rise in CEA and the emergence of new metastasis in liver and lung. The entire anti-cancer treatment was stopped in November 2005. At this stage the CEA levels were 19.5 ug/l. In January 2006 CEA levels increased further to 26 ug/l. At this stage the compassionate treatment with Wormwood preparation as cited in invention 1 was initiated after the patient gave his informed consent. When the treatment with the invention 1 was started, there were at least five small metastasis of the size 3-5 mm in both lungs. Ultrasonic contrast tests of the liver revealed in segment VI three metastasis of the size 2-3 cm and the CEA levels were 26 ug/l. Four months later, the CEA dropped to 16, the metastasis in liver and lungs were reduced by 50% and the patient was feeling subjectively well.

### Example 2

Powdered dried A. annua to treat multiple myeloma patients whose disease recurred in spite of chemotherapy and became resistant to available conventional treatments.

Invention 2: Powdered dried A. annua pressed in 750 mg tablets given 3×4 tabs/day to treat multiple myeloma patients whose disease recurred in spite of chemotherapy and became resistant to available conventional treatments.

Case Reports: Four multiple myeloma (MM) patients exhibiting β-2 microglobulin above 3 mg/l—two of them with and two without deletion of chromosome 13 by fluorescence in situ hybridization (FISH) analysis, showing recurrence and progression of their MM disease in spite of previous treatment with vincristine+doxorubicin+melphalan combinations were recruited to receive A. annua (invention 2) treatment after their informed consent was obtained. All patients received a new standard cycle of chemotherapy standard cycle of chemotherapy as follows:
Continuous intravenous infusion of 0.4 mg vincristine/m2 body surface for 24 hrs
Continuous intravenous infusion of 9 mg of doxorubicin/m2 over 24 hrs
Oral administration of 40 mg of dexamethasone

All three anti cancer drugs were administered for four days. The cycle was repeated after 3 weeks interval. In total there were 3 cycles. In addition all patients received 3×4 tablets of invention 2. The invention 2 was given for four months, before (2 weeks), during (9 weeks) and after (4 weeks) the anti-cancer cycles). The results are summarized as follows: One “Complete Remission” (=No detectable para-proteins in serum and urine and 5% or less plasma cells one patient). One “Good partial response” (=Decrease of 90% in serum para-proteins two patients). Two “Satisfactory partial response” (=Decrease of 50% in para-proteins in serum or 90% decrease in Bence Jones proteins one patient).

### Example 3

Distilled preparation of A. absinthium and large cardamom to treat progressing uveal melanoma stage IV.

Invention 3: Distilled extract (Tinctures) of A. absinthium and large cardamom 3 ml (equivalent to 9 g powdered herb/day) three times a day to treat progressing uveal melanoma stage IV.

Case Report: A female born in 1942, was diagnosed with uveal melanoma in September 1999. Her left eye was enucleated, and subsequently it was under control without pathologic findings until February 2002 when distant metastases were first diagnosed. Upon examination, she was otherwise in good general health. A CT-scan revealed multiple lung metastases on both sides, as well as multiple liver metastases. Chemotherapy with Dacarbazine (DTIC) was initiated at a concentration of 850 mg per m2 body surface.

After 3 months and 4 courses, a CT-staging showed progressive disease, therefore DTIC was stopped and chemotherapy with Fotemustine at a concentration of 100 mg per m2 body surface was started. After 3 months and 4 courses, the disease was moderately progressing, although a CNS metastasis was diagnosed and had to be treated by stereotactic irradiation, and 3 new skin metastases on the scalp were detected and surgically removed. After 3 additional courses, the known metastases were slightly increasing in size. Since no new organs became involved, we proceeded with Fotemustine therapy. However, after another 3 courses, a further progression was diagnosed in August 2002 with known metastases increasing in size and number, as well as the involvement of another organ, the pancreas. Due to a lack of standard regimens after 2 chemotherapies had failed, we started a compassionate treatment with daily invention 3 (3 times daily, 2 ml of the extract p.o.) in combination with Fotemustine after receiving the informed consent of the patient. The first staging after 3 courses of Fotemustine in combination with invention 3 showed a mixed response 3 months after the initiation of this therapy mode.

Although there were no significant changes in the liver, lung metastases were decreasing in number and size and there was no evidence of new metastases in visceral organs or on the skin. After this phase of relative stabilization of disease progression, the subsequent staging demonstrated significant progress. We therefore changed the chemotherapy to DTIC, but continued with invention 3. Two years after entry into stage IV as defined by AJCC (14) the patient was in good health. Throughout the combination therapy, there were no additional side effects other than those caused by chemotherapy alone, such as nausea, vomiting and bone marrow insufficiency leading to anaemia, leukopenia and thrombopenia with the expected latency after the chemotherapy courses.

### Example 4

Extract of ginger root and A. capillaris to treat regional colitis—an autoimmune disease.

Invention 4: Liquid extract of A. capillaris and ginger root=3 ml three times a day (equivalent to 9 g powdered herba) to treat regional colitis—an autoimmune disease.

Ten patients suffering from regional colitis (Crohn's Disease, CD) which is an autoimmune disease refractory to their chemotherapy were given in addition to their basic chemotherapy invention 4 for 6 weeks. Minimum score of 200 on Crohn's Disease Activity Index (CDAI) was required at baseline. Patients who received infliximab or similar immune-modulators in the past were excluded from the trial. TNF-α levels in serum were measured at baseline, after three and six weeks. All concomitant CD medications was maintained at the baseline dose levels. Results: Average serum TNF-α levels fell from 16.5±3.5 pg/ml at week 0 to 8.0±2.5 pg/ml at week 6. On the clinical side, average CDAI fell from 18.275±15 to below 175±12 with almost complete remission of symptoms in 6 patients (CDAI below 170). IBD-Questionnaire also reflected accelerated clinical response with ginger. Conclusions: The use of a combination of ginger and A. capillaris in CD and other TNF-α targeting auto-immune diseases such as lupus, myositis, glomurelonephritis, and similar are justifiable.

### Example 5

Powdered dried combination of A. vulgaris and large cardamom to treat rheumatic polyarthritis—an autoimmune disease.

Invention 5: The following ingredients by mass of active ingredients were beneficial:

| | |
|---|---|
| Artemisa vulgaris (common wormwood): | 60% |
| Ginger root (Zingiber officinale): | 20% |
| Large Cardamom (Amomum subulatum): | 20% |

The powdered herbs were filled in gelatine capsules of the size 00 so as to give a final weight of 450 mg/capsule.

The ingredients of the composition have overlapping activity, and they act together synergistically when added in the particular proportion suggested in accordance with the invention. Studies by the inventor have shown that the beneficiated extracts of the various plants disclosed above exhibit biological activity far greater than the corresponding quantity of crude powder from individual plants which the beneficiated extract is derived. For instance, 100 ml of extract, obtained from 100 gm of crude mix of 60 gm of wormwood+20 g long cardamom++20 g ginger, provides the equivalent pharmacological and biological activity corresponding to 1000 gs of crude powder of wormwood on cell and animal models.

The process disclosed in accordance with this invention creates a composition which retains the natural proportions of the active ingredients of a pharmacologically beneficial plant and at the same time provides a convenient dosage formulation. An additional benefit is that, the process of beneficiation makes the consumption of plant economical, since there is a more than ten fold increase in the activity equivalence and a lesser quantity of crude powder is required to provide the required therapeutic dosage of a pharmacologically active plant.

Accordingly, for the treatment of rheumatic diseases, more precisely for the treatment of Rheumatoid Arthritis powdered dried herbal combination according to invention 5 was prepared. The recommended dose was 9 capsules a day in 3 divided doses for a period of not less than three months. The results are summarized below:

| Number of Patients | | |
|---|---|---|
| N = 5 | Baseline | After 3 months |
| Severity of Joint Pains | Moderate to severe | Almost none |
| Morning Stiffness | Moderate to severe | Almost none |
| Joint Score | 4 | 1 |
| Ritche Articular Index | 4 | 1 |
| ESR | 40/hr | 8/hr |

Typically, the composition according to the invention for the treatment of rheumatic disease also contains ginger powder to enhance the clinical efficacy. The following ingredients by mass of active ingredients were also beneficial:

| | |
|---|---|
| Artemisa asiatica (asian wormwood): | 60% |
| Ginger (Zingiber officinale): | 20% |
| Large Cardamom (Amomum subulatum): | 20% |

This composition also showed general beneficial effects on other laboratory variables, such as rheumatoid factor which is a more specific immunological variable to monitor and in tests conducted on patients. The composition removed rheumatoid factor from the serum in moderate proportions of sero-positive arthritis patients and 30-50% of these patients received considerable long protection (12 months) against degenerative and destructive processes which commonly occur in such diseases resulting in morbidity. The composition in long-term treatment of minimum six months duration also showed beneficial radiological changes. Minimum duration of treatment with the composition for considerable clinical benefits is three months and dose will be six tablets or capsules or equivalent quantity of oral liquid three times a day preferably after food intake.

### Example 6

Powdered dried combination of A. annua, ginger and large cardamom to treat IgA Nephropathy.

Invention 6: Each capsule of the size 00 contained powdered dried powder of

| | |
|---|---|
| Artemisa annua (sweet wormwood): | 300 mg |
| Ginger (Zingiber officinale): | 40 mg |
| Large Cardamom (Amomum subulatum): | 40 mg |
| Rose petals as inert herb | 80 mg |
| Total weight of the capsule: | 460 mg |

Case Report: In the urine test of a young female patient born on Apr. 20, 1988 pathological levels of protein was discovered. Histology of kidney biopsy confirmed the diagnosis of IgA-Nephritis with mesegioproliferative glomerulonephritis in August 2006. All other immune relevant laboratory findings such as ANA, ANCA, anti-ds-DNS-Antibodies, C3, C4 and antistreptolysin titer were in normal range. Hepatitis B and C serology was normal. Electrophoresis of urine sediment revealed IgA proteins. Pathological was the amount of proteins in urine and Protein/Creatinine quotient. The treatments given are summarized in the following table:

| | | Creatinine/ | |
|---|---|---|---|
| | Protein | Protein | |
| Dates | in urine | quotient | Treatment |
| 20.09.2005 | 179 | 865 | Ramipril 5 mg |
| 28-09.2005 | 380 | 2603 | Ramipril 5 mg + Valsartan 80 mg |
| 30.10.2005 | 535 | 2149 | Ramipril 5 mg + Valsartan 80 mg |
| 20.11.2005 | 81 | 466 | Ramipril 5 mg + Valsartan 80 mg |
| 20.12.2005 | 53 | 269 | Ramipril 5 mg + Valsartan 80 mg |
| 13.02.2006 | 314 | 1328 | ACE + Diovan 80 |
| 01.03.2006 | 515 | 1966 | Ramipril 5 mg + Valsartan |
| | | | 80 mg + Invention |
| 14.03.2006 | 197 | 1126 | Ramipril 5 mg + Valsartan |
| | | | 80 mg + Invention |
| 10.04.2006 | 46 | 297 | Ramipril 5 mg + Valsartan |
| | | | 80 mg + Invention |

The table demonstrates the curing effect of the invention in IgA Nephropathy. There was moderate recovery to ACE treatment with Ramipril 5 m/day initially, but soon after the condition of the patient deteriorated. As the table shows, the progress of the disease was rapid. Diovan 80 mg/day were added to Ramipril. With this combination there was a temporary recovery. Soon after the disease started to progress again. On Jan. 3, 2006 we started with the invention described under example The recovery was rapid and impressive. It is for the first time that a progressing disease of IgA Nephropathy was brought under control by a herbal preparation.

### Example 7

Powdered dried combination of A. absinthium, ginger and large cardamom to treat Gioblastoma stage IV

Invention 7: Each capsule of the size 00 contained powdered freeze dried powder of

| | |
|---|---|
| Artemisa absinthium (wormwood): | 290 mg |
| Ginger (Zingiber officinale): | 40 mg |
| Large Cardamom (Amomum subulatum): | 40 mg |
| Rose petals as inert herb | 80 mg |
| Total weight of the capsule: | 450 mg |

An estimated 18,500 brain and spinal cord cancers were diagnosed in 2005 in the United States, at least 85 percent of which will be brain tumors. Approximately 12,760 people died from brain and spinal cord cancers this year, accounting for 1.4 percent of all cancers and 2.4 percent of all cancer deaths. The average survival for Glioblastoma is approximately one year. Glioblastom is one of the most deadly and fastest growing brain cancers. Tumors that originate in and metastasise to the brain are among the most difficult to treat. For Glioblastoma and there is no satisfactory treatment. Two types of imaging tests—magnetic resonance imaging (MRI) and computed tomography (CT)—mainly are used to diagnose brain tumors.

A number of research centers are testing novel agents, alone or in combination, that inhibit angiogenesis, cell proliferation and tumor cell migration. While surgery, radiation therapy and chemotherapy remain the mainstays of treatment for these cancers, they are far from perfect. Surgery can be difficult and risky if the tumor is near a vital portion of the brain. Chemotherapy agents have to find ways past the blood-brain barrier. Radiation can damage healthy tissue. Surgery is the most common treatment for brain cancer (and frequently the only treatment for benign brain tumors). It is often extremely difficult to remove a brain cancer completely, given that it usually invades other, normal brain tissue as well. Only three drugs are approved by the U.S. Food and Drug Administration for the treatment of malignant gliomas: BCNU, CCNU and temozolomide. All three have very limited success.

Case Report: In the brain of a 44 years old female patient, Glioblastoma was detected in early 2005. In spite of neuro-surgical removal the cancer was expanding in vital regions of the brain. The patient was put on several cycles of BCNU and CCNU and temozolomide treatment at Baltimore Medical center, Maryland, USA. No signs of tumour regression were observed in routine MRI and CT tests. Finally a compassionate treatment was initiated with invention 7 in combination with temozolomide. Soon after, the appetite of the patient returned, four months later the patient could move around without help, and after six months the tumour size as shown by MRI and CT was reducing. After one year, the patient is still alive and in good health.

### Example 8

Powdered dried combination of A. absinthium, ginger and large cardamom to treat aggressive Non-Hodgkin Lymphoma

Hodgkin and Non-Hodgkin Lymphomas (NHL) are cancers of the lymph system, when untreated lead to death. Radiation is not effective. They are usually treated with a combination of Cyclophospahmid, Vincristin, Prednison and Doxorubicin, a standard treatment called CHOP-14. In spite of this intensive chemotherapy the rates of re-appearance of disease are high and the death comes soon irrespective of the age of the patients. In our example we treated 5 patients with aggressive NHL with treatment failure. The above cited combination of invention in example 7 was given in capsule form (3×3 caps/day) for nine month in combination with another four rounds of CHOP at 14-day interval. The staging of the cancer after three months revealed almost complete remission of the disease with normal blood cells and normal bone marrow biopsy. Only spleen and liver were still enlarged due to the past progression of the disease. Two young Hodgkin patients were treated similarly and showed improvements.

### Example 9

Powdered dried combination of A. absinthium, ginger and large cardamom to counteract weight loss and improve nutrition in cancer patients according to the invention 7 in example 7.

Fifty-two consecutive patients with advanced cancer were prospectively randomized to either take normal nutrition (NN; 24 patients) or invention 7 plus normal nutrition (PN+; 26 patients). Body weight, body mass index (BMI), and caloric intake were assessed, and haemoglobin (g/dL) and serum albumin (g/L) were measured. Body composition was assessed by body impedance analysis (BIA), and QOL was evaluated by European Organization for Research and Treatment of Cancer (EORTC) QLQ-C30 questionnaire every 6 weeks. Results: No significant differences were evident at baseline between the 2 groups for age, gender, medical diagnosis, weight, BMI, or QOL. A statistically significant difference in mean BMI was observed by week 48 for the PN+ group (PN+=21.9, NN−=20.5, p=0.0149), by week 6 in mean body cell mass (PN+=55%, NN−=50.1%, p<0.001), mean albumin (PN+=40.2 g/L, NN−=36.2 g/L, p=0.015), mean QOL (PN+=55.7, NN−=50.9, p=0.035). The cumulative survival rate was significantly greater in the PN+ group (p<0.0001). Conclusions: According to the positive effect of supplemental PN+ on survival, body composition, and QOL, invention in example 7 is of great benefit for cancer patients suffering from malnutrition and weight loss. At present there is no other treatment available to counteract the weight loss and nutritional deficiency in cancer patients. This weight gain is not the result of better functioning of gastrointestinal digestive system or gallbladder functioning, but is the result of suppression of those compound released by cancer cell that are responsible for appetite loss. This conclusion is supported by the fact that other appetite increasing herbs such as the cynara scolynus, curcuma roots, anis, chelidonium majus, peumus boldus (boldo leaves) were unable to produce this effect.

1. Method to counteract the weight loss and nutritional deficiency of cancer patients, and to treat Hodgkin and Non-Hodgkin lymphomas, autoimmune diseases, IgA-Nephropathy (glomerulonephritis) and human cancers with a herbal preparation containing Artemisia.

2. A method of claim 1 whereby the herbal preparation containing Artemisia is selected from the group consisting of Artemisia absinthium, Artemisia annua, Artemisia vulgaris and Artemisia capillaris or any bitter or aromatic herb or shrub of the genus Artemisia of the family Asteraceae, distributed throughout many parts of the world.

3. A method of claim 1 wherein the said autoimmune disease involves joints, kidneys, blood vessels, intestine, eyes, lungs, nerves, muscles and mucous membranes.

4. A method of claim 1 whereby the said autoimmune disease is selected from the group consisting of rheumatic diseases, lupus, colitis, sarkoidosis, arteritis, amyloidosis, kidneys, Behcet and Reiter's disease.

5. A method of claim 1 wherein the said cancer is selected from the group consisting of breast, brain, lung, stomach, colon, rectum, kidney, ovaries, prostate gland, blood, cartilages and bones.

6. A method of claim 1 whereby the conventional standard therapy consists of chemotherapy, immune-therapy, surgery, radiation or any combination of these.

7. A method of claim 1 and claim 2 in which 10 to 90% by weight of fruits of large cardamom (Amomum subulatum) is added.

8. A method of claim 1 and claim 2 in which 10 to 90% by weight of ginger root (Zingiber officinale rhizomes) is added.

9. A method of claim 1 and claim 2 in which 10 to 80% by weight of ginger root (Zingiber officinale rhizomes) and 10 to 80% by weight of large cardamom (Amomum subulatum) are added.

10. A method of claim 1 through 9 wherein the amount of herbs is sufficient to suppress the progress of the disease.

11. A method of claim 1 through 9 in which the invention is prepared by grinding, solvent extraction or distillation.

12. A method of claim 1 through 9 in which inert pharmaceutical carrier is added consisting of an inorganic salt, organic molecule or a herb.

13. A method of claim 1 through 12 whereby the herbal preparation is applied in combination with the conventional standard therapy.

14. A method of claim 1 through 13 whereby the invention is applied to modulate the immune system of the human body.
